(19) United States
(12) Patent Application Publication
(10) Pub. No.: US 2023/0270902 A1
(43) Pub. Date: Aug. 31, 2023

(54) SYSTEM AND METHOD FOR AUTOMATED VEHICLE SANITATION

(71) Applicant: Continental Automotive Systems, Inc., Auburn Hills, MI (US)

(72) Inventors: Rujiao Yan, Oberursel (DE); Nils Hamacher, Darmstadt (DE); Matthew Donald Berkemeier, Beverly Hills, MI (US); Jacob Andrew LaPonse, Waterford, MI (US)

(73) Assignee: Continental Automotive Systems, Inc., Auburn Hills, MI (US)

(21) Appl. No.: 17/652,925

(22) Filed: Feb. 28, 2022

(51) Int. Cl. A61L 2/20; A61L 2/24

(52) U.S. Cl. CPC A61L 2/208; A61L 2/24; A61L 2202/14; A61L 2202/25; A61L 2202/15

(57) ABSTRACT

A process for disinfecting a vehicle includes identifying an end of operations of the vehicle, dispersing a nebulized disinfectant into the internal compartment, displaying a progress bar such that the progress bar is visible external to the compartment, wherein the progress bar is indicative of a time remaining in the duration of a disinfectant process, and allowing access to the internal compartment at an expiration of the time remaining in the disinfectant process.

### TECHNICAL FIELD

The present disclosure relates generally to vehicle maintenance and sanitation, and more specifically to an automated system and method for sanitizing a vehicle after use.

### BACKGROUND

Shared vehicles such as public transportation (alternately referred to as mass transportation), rental cars, rental trucks, and the like have large turnover between passengers and operators within a short period of time. Many people care about the passenger and operator spaces being maintained in a clean and sanitary manner in order to prevent the spread of contagions. Manual sanitation between operations of the vehicles requires notification to a maintenance employee, and time for the maintenance employee to arrive and sanitize the vehicle. In addition to the time cost, employing a maintenance worker to sanitize passenger and operator spaces in between vehicle operations imposes a substantial monetary cost and management burden.

### SUMMARY OF THE INVENTION

In one exemplary embodiment a process for disinfecting a vehicle includes identifying an end of operations of the vehicle, dispersing a nebulized disinfectant into the internal compartment, displaying a progress bar such that the progress bar is visible external to the compartment, wherein the progress bar is indicative of a time remaining in the duration of a disinfectant process, and allowing access to the internal compartment at an expiration of the time remaining in the disinfectant process.

In another example of the above described process for disinfecting a vehicle identifying the end of operations includes receiving a video feed of an internal compartment of a vehicle at a vehicle controller and initiating the disinfectant process in response to identifying a lack of occupants in the video feed using image analysis.

In another example of any of the above described processes for disinfecting a vehicle the image analysis includes identifying a lack of faces within the video feed using a facial recognition module.

In another example of any of the above described processes for disinfecting a vehicle the nebulized disinfectant includes hydrogen peroxide.

In another example of any of the above described processes for disinfecting a vehicle the disinfectant process is characterized by a lack of ultra-violet (UV) disinfectant).

Another example of any of the above described processes for disinfecting a vehicle further includes locking each entrance to the internal compartment for the duration of the disinfectant process using the vehicle controller.

In another example of any of the above described processes for disinfecting a vehicle the vehicle is a shared passenger vehicle.

In another example of any of the above described processes for disinfecting a vehicle the vehicle is a mass transportation vehicle.

In one exemplary embodiment a vehicle includes an internal occupant compartment, at least one disinfectant dispersal unit within the internal occupant compartment, a disinfectant storage tank fluidly connected to the at least one disinfectant dispersal unit, at least one occupant sensor configured to detect occupants within the internal occupant compartment, and a controller controllably coupled to the at least one disinfectant dispersal units, the disinfectant storage tank and the at least one occupant sensor, the controller being further configured to disperse a nebulized disinfectant from the disinfectant storage tank into the internal occupant compartment using the at least one disinfectant dispersal unit in response to the vehicle ending a vehicle operation and the at least one occupant sensor detecting no occupants within the internal occupant compartment.

Another example of the above described vehicle further includes at least one vehicle access component controllably coupled to the controller and wherein the controller is configured to prevent vehicle access through the vehicle access component for a duration of a disinfectant dispersal process.

In another example of any of the above described vehicles the disinfectant storage tank includes a storage tank portion and a disinfectant nebulizer portion.

Another example of any of the above described vehicles further includes at least one display visible from an exterior of the internal occupant compartment the display being communicatively coupled to the controller.

In another example of any of the above described vehicles the controller is configured to cause the display to display a remaining duration of the disinfectant dispersal.

In another example of any of the above described vehicles the vehicle is a passenger vehicle.

In another example of any of the above described vehicles the vehicle is a mass transit vehicle.

In another example of any of the above described vehicles the at least one occupant sensor includes a video camera, and the controller includes a facial recognition module configured to identify faces in an output of the video camera.

In another example of any of the above described vehicles the controller is configured to determine that the at least one occupant sensor detected no occupants in response to the facial recognition module detecting no faces in the output of the video camera.

In another example of any of the above described vehicles a disinfectant stored in the storage tank is hydrogen peroxide.

These and other features of the present invention can be best understood from the following specification and drawings, the following of which is a brief description.

### BRIEF DESCRIPTION OF THE DRAWINGS

FIG. 1 illustrates an exemplary interior view of a shared vehicle including an automated sanitization system.

FIG. 2 schematically illustrates a disinfectant dispersal system for use within a shared vehicle.

FIG. 3 schematically illustrates an exemplary sensor view of an interior of a shared vehicle.

FIG. 4 illustrates a method for activating an automated sanitization system for a vehicle.

FIG. 5 illustrates a method for disinfecting a vehicle using the automated sanitization system for a vehicle.

The embodiments, examples, and alternatives of described in the claims and in the following description and drawings, including any of their various aspects or respective individual features, may be taken independently or in any combination. Features described in connection with one embodiment are applicable to all embodiments, unless such features are incompatible.

### DETAILED DESCRIPTION

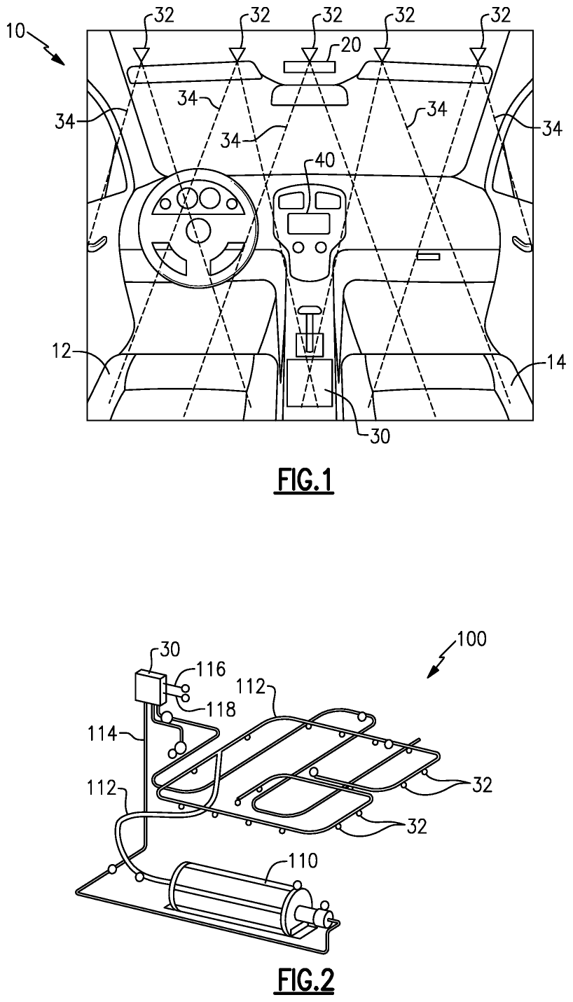

FIG. 1 schematically illustrates an internal portion of a vehicle 10 including an operator seat 12 and a passenger seat 14. A sensor system 20, such as a camera, is positioned with a sensing field of view that includes each of the operator seat 12 and the passenger seat 14. In examples including a larger occupant space, such as a bus or train car, the sensor system 20 includes a field of view encompassing all of the potential occupant spaces and the sensor system 20 can include multiple sensors disposed about the potential occupant spaces.

Also included in the vehicle is a sanitization controller 30, configured to control multiple sanitization dispersal units 32 within an automated sanitization system. Each of the multiple sanitization dispersal units 32 provides a corresponding zone 34 in which the disinfectant is directly dispersed. Gaseous dispersion of the disinfectant (e.g., a nebulized disinfectant) ensures that even areas not in one of the direct zones 34 are still disinfected. Inclusion of multiple units 32 decreases the time that it takes for the gaseous disinfectant to disperse throughout the vehicle and contact all the surfaces. In an example of a passenger vehicle, the units 32 may be placed on the upper sides with one unit 32 on each front side and a unit 32 in the middle of the rear. On a train, or other multi carriage vehicle, the units 32 may be placed one per carriage. In alternative examples alternative positioning could be utilized to similar effect. In addition to the inclusion of the direct zones 34,

In some examples, such as a shared passenger vehicle, the controller 30 is a component of a general vehicle electronic control unit. In alternative examples, such as mass transit public transportation, the sanitization controller 30 is a dedicated controller and is not included in a general control system for the vehicle. One of skill in the art will appreciate that the passenger vehicle and mass transit vehicles are non-limiting examples and mass transit vehicles can utilize a general controller including a sanitization component or shared passenger vehicles can include dedicated sanitation controllers.

The sanitization controller 30 is communicatively coupled to the sensor system 20, and is able to detect the presence of passengers and/or operators within the internal portion of the vehicle 10 based on the detections of the sensor system 20. At the end of a vehicle operation (e.g., when a shared passenger vehicle is being transitioned to a new operator, or when a mass transit vehicle is offboarding passengers at a stop), the sensor system 20 detects when the internal portion of the vehicle 10 is emptied of passengers. In some examples, the sanitization controller 30 can also include a verification system that provides a request for confirmation that the internal portion is empty. In such an example, once the sensor system 20 determines the internal portion is empty, an operator confirms the sensor reading and the sanitization controller 30 determines that the internal portion is empty.

Once the sanitization controller 30 determines that the internal portion of the vehicle 10 is empty, the sanitization controller 30 activates the dispersal units 32 which disperse a gaseous disinfectant via a nebulization process to fill the compartment.

In one example, the disinfectant is a nebulized hydrogen peroxide sanitizer. The hydrogen peroxide nebulizer uses a steam generator to disperse a steam form of hydrogen peroxide as a gas into the internal portion of the vehicle 10. The gaseous nature of the hydrogen peroxide ensures that the hydrogen peroxide will expand to fill the entire space of the internal portion of the vehicle 10. Filling the entire space ensures that the hydrogen peroxide contacts every surface within the internal portion, ensuring complete disinfecting. In addition, the gaseous nature allows for the disinfectant to break down faster than a liquid form and decreases the time for the internal portion of the vehicle to be usable again.

The exemplary system uses hydrogen peroxide as a disinfectant because hydrogen peroxide breaks down into water and oxygen, both of which are safe for contact with people or other living beings (e.g., pets). In alternative examples, similar disinfectant compounds can be utilized given proper ventilation and breakdown time. In order to prevent degradation of plastic components, as well as other light sensitive components within the internal portion of the vehicle 10, and ensure that the entire compartment is disinfected including areas where a light path may be obstructed, the disinfectant process does not utilize ultra-violet (UV) light disinfecting.

In some examples, the controller 30 is connected to door locks or other access restricting elements, and the controller 30 can automatically prevent access to the internal portion of the vehicle 10 by locking the doors or preventing access through the access restricting elements. In addition to restricting access, the controller 30 can be connected to a window actuator such that the controller can cause the windows, or other openings, to be opened and closed and ensure that the compartment is at least primarily gastight. As used herein, “primarily gastight” refers to a magnitude of sealing which allows a sufficiently small amount of the disinfectant gas to escape that the gas fills the entire compartment rather than escaping before the compartment is filled. Alongside access restriction, the controller 30 can cause one or more screens 40 to display a timer or other countdown that allows a user attempting to access the internal portion of the vehicle 10 to know how long until the disinfectant process has been completed and the internal portion of the vehicle 10 is ok to enter. In particular, the controller 30 can cause displays (screens 40) that are visible to individuals outside of the vehicle and may be waiting to enter to display a progress bar, countdown, or similar counter indicating when the individuals will be able to enter.

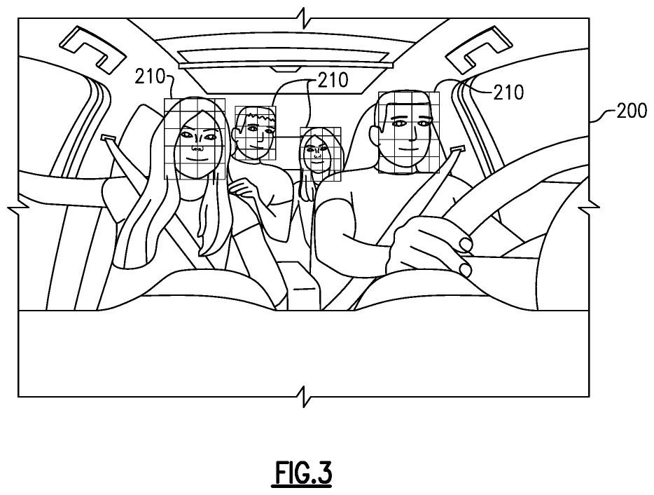

With continued reference to FIG. 1, FIG. 2 illustrates an exemplary disinfectant dispersal system 100 for use in the vehicle 10 of FIG. 1. The disinfectant dispersal system 100 includes the dispersal units 32 and the controller 30 of FIG. 1 as well as a storage tank/nebulizer 110. The storage tank/nebulizer 110 is connected to each of the dispersal units 32 via a fluid connection 112 and includes an internal apparatus for nebulizing the disinfectant stored in the storage tank/nebulizer 110. The controller 30 includes control connections 112, 114 that allow the controller 30 to control operations of the storage tank/nebulizer 110 and the dispersal units 32. In some examples the controller 30 control connections 116 that are also connected to displays, such as entertainment systems, and access elements, such as locks, in order to control access to the interior portion of the vehicle 10. The dispersal units 32 and the storage tank/nebulizer 110 can be controlled using any conventional control methodology.

Another set of communication connection 118 connects the controller 30 to the sensor system 20. The controller 30 receives the sensor information from the sensor systems 20 and the controller 30 analyzes and interprets the information to identify occupants within the internal portion of the vehicle 10. While illustrated as a single line connection 118, one of skill in the art will appreciate that the representative illustration can include multiple connections to multiple sensors and sensor types, and the controller 30 can be configured to combine the sensor details to synthesize the presence of one or more person within the vehicle according to any conventional data interpretation techniques. By way of example, the sensors can include an image feed, weight sensors in the seats, hand sensors in a steering wheel, etc.

With continued reference to FIGS. 1 and 2, FIG. 3 illustrates an exemplary sensor view 200. The sensor view 200 is generated by a rear facing video camera sensor in the sensor system 200, with the rear facing video camera sensor being positioned to have a field of view of all the occupants within the internal portion of the vehicle 10. The controller 30 includes a facial identification module that analyzes the video feed to identify the presence of faces 210 using any conventional facial identification software. The controller 30 is configured to determine that any time a face 210 is present within the field of view, an occupant is present in the internal portion of the vehicle 10, and the disinfectant system should not be activated. In alternative examples, the video based facial detection can be replaced with an infrared sensor configured to detect the presence of people and/or pets within the vehicle, and the detection can be operated similarly. In yet further examples, the sensor detection can be supplemented with an operator confirmation, as described above.

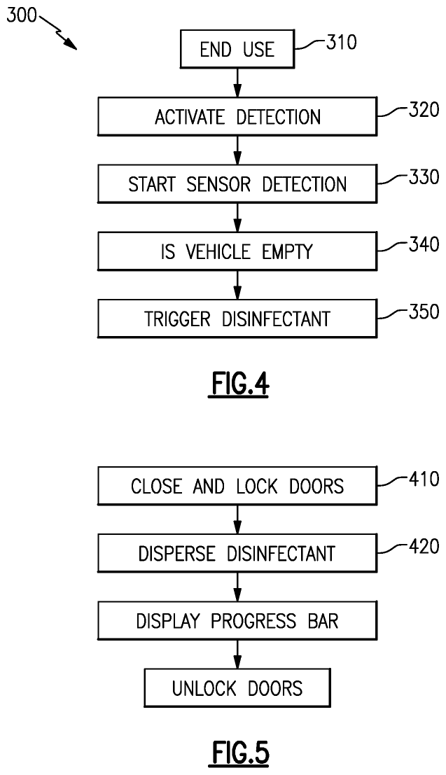

With continued reference to FIGS. 1-3, FIG. 4 illustrates a method 300 for activating the automatic disinfectant system 100 and automatically disinfecting the internal portion of the vehicle 10. Initially the controller 30 detects an end of vehicle operations in an “End Use” step 310. The end of the use can be detected via any number of ways including a mass transit vehicle arriving at a destination, a rental period of a shared passenger vehicle ending, or any other means.

Once the end of the operation is detected, the data from the sensor system 20 is reviewed by the controller 30 in an “Activate Detection” step 320. In alternative examples, the user detection process can be operated constantly and the activate detection step 320 can be omitted as the process is already occurring. In either case, the method proceeds to a “Start Sensor Detection” step 330, where the controller analyzes the data from the sensors to determine the presence of occupants within the internal portion of the vehicle 10. In the example illustrated above, the sensor data is a video feed, and the feed is analyzed for the presence of faces. In alternative examples, seat weight sensors can detect when occupants stand up, and all seats being empty can be indicative of the internal portion of the vehicle 10 being empty.

During the detection process, the controller 30 runs an “Is Vehicle Empty” check 340. When the controller returns a yes to the check based on the sensor detection (i.e., when the sensors detect no occupants in the internal portion of the vehicle), the process activates the disinfectant process in a “Trigger Disinfectant” step 350. In the trigger disinfectant step 350, the controller 30 operates the disinfectant system 100 to disinfect the internal portion of the vehicle 10.

With continued reference to FIGS. 1-4, FIG. 5 illustrates an example disinfectant process that occurs in response to the controller triggering the disinfectant process. Initially in the disinfectant process, access to the interior portion of the vehicle 10 is restricted for the duration of the disinfecting process in a “Close and Lock Doors” step 410. When the controller 30 is connected to vehicle access systems like door locks, and windows, the controller closes and locks the doors. In other examples, where alternative vehicle access systems, such as train platform access, may be connected to the controller 30 and the alternative systems used to prevent access during the disinfecting process.

Once access to the internal portions of the vehicle 10 has been prevented, the controller causes the hydrogen peroxide, or similar disinfectant, to be dispersed into the internal portion of the vehicle in a “Disperse Disinfectant” step 420. The disinfectant is dispersed by nebulizing the disinfectant in the storage tank/nebulizer 100 and providing the steam to the dispersal units 32. The controller 30 is configured to open the dispersal units and meter out a sufficient disinfectant through the dispersal units 32 to allow for all surfaces to be contacted by a sufficient amount of the nebulized disinfectant.

During the dispersal process, the controller 30 communicates with at least one exterior facing display or display visible from the exterior of the vehicle and causes the display to show the countdown, allowing individuals waiting to access the vehicle to know a waiting time until they can access the disinfected vehicle. The countdown can be a numerical countdown, a progress bar countdown, or any other indicator (or combination of indicators) that intuitively communicates the remaining time until the compartment is sanitized.

It is further understood that any of the above described concepts can be used alone or in combination with any or all of the other above described concepts. Although an embodiment of this invention has been disclosed, a worker of ordinary skill in this art would recognize that certain modifications would come within the scope of this invention. For that reason, the following claims should be studied to determine the true scope and content of this invention.

1. A process for disinfecting a vehicle comprising:
identifying an end of operations of the vehicle;
dispersing a nebulized disinfectant into the internal compartment;
displaying a progress bar such that the progress bar is visible external to the compartment, wherein the progress bar is indicative of a time remaining in the duration of a disinfectant process; and
allowing access to the internal compartment at an expiration of the time remaining in the disinfectant process.

2. The process of claim 1, wherein identifying the end of operations includes receiving a video feed of an internal compartment of a vehicle at a vehicle controller and initiating the disinfectant process in response to identifying a lack of occupants in the video feed using image analysis.

3. The process of claim 2, wherein the image analysis includes identifying a lack of faces within the video feed using a facial recognition module.

4. The process of claim 1, wherein the nebulized disinfectant includes hydrogen peroxide.

5. The process of claim 1, wherein the disinfectant process is characterized by a lack of ultra-violet (UV) disinfectant).

6. The process of claim 1, further comprising locking each entrance to the internal compartment for the duration of the disinfectant process using the vehicle controller.

7. The process of claim 1, wherein the vehicle is a shared passenger vehicle.

8. The process of claim 1, wherein the vehicle is a mass transportation vehicle.

9. A vehicle comprising:
an internal occupant compartment;
at least one disinfectant dispersal unit within the internal occupant compartment;
a disinfectant storage tank fluidly connected to the at least one disinfectant dispersal unit;
at least one occupant sensor configured to detect occupants within the internal occupant compartment; and
a controller controllably coupled to the at least one disinfectant dispersal units, the disinfectant storage tank and the at least one occupant sensor, the controller being further configured to disperse a nebulized disinfectant from the disinfectant storage tank into the internal occupant compartment using the at least one disinfectant dispersal unit in response to the vehicle ending a vehicle operation and the at least one occupant sensor detecting no occupants within the internal occupant compartment.

10. The vehicle of claim 9, further comprising at least one vehicle access component controllably coupled to the controller and wherein the controller is configured to prevent vehicle access through the vehicle access component for a duration of a disinfectant dispersal process.

11. The vehicle of claim 9, wherein the disinfectant storage tank includes a storage tank portion and a disinfectant nebulizer portion.

12. The vehicle of claim 9, further comprising at least one display visible from an exterior of the internal occupant compartment the display being communicatively coupled to the controller.

13. The vehicle of claim 12, wherein the controller is configured to cause the display to display a remaining duration of the disinfectant dispersal.

14. The vehicle of claim 9, wherein the vehicle is a passenger vehicle.

15. The vehicle of claim 9, wherein the vehicle is a mass transit vehicle.

16. The vehicle of claim 9, wherein the at least one occupant sensor includes a video camera, and the controller includes a facial recognition module configured to identify faces in an output of the video camera.

17. The vehicle of claim 16, wherein the controller is configured to determine that the at least one occupant sensor detected no occupants in response to the facial recognition module detecting no faces in the output of the video camera.

18. The vehicle of claim 9, wherein a disinfectant stored in the storage tank is hydrogen peroxide.
